(19) United States
(12) Patent Application Publication
(10) Pub. No.: US 2006/0009666 A1
(43) Pub. Date: Jan. 12, 2006

(54) Hydrogenation of aromatics and olefins using a mesoporous catalyst

(75) Inventors: Bala Ramachandran, Bethlehem, PA (US); Martin Kraus, Worms (DE); Zhiping Shan, Austin, TX (US); Philip J. Angevine, Woodbury, NJ (US)

Correspondence Address: DILWORTH & BARRESE, LLP, 333 EARLE OVINGTON BLVD., UNIONDALE, NY, 11553, US

(73) Assignee: ABB Lummus Global, Inc., Bloomfield, NJ (US)

(21) Appl. No.: 11/032,808

(22) Filed: Jan. 10, 2005

Related U.S. Application Data
(63) Continuation-in-part of application No. 10/886,993, filed on Jul. 8, 2004.

(51) Int. Cl. C07C 7/163; C07C 5/10; C07C 5/03; C10G 69/00; C10G 65/12

(52) U.S. Cl. 585/258; 585/266; 585/275; 208/259; 208/58

(57) ABSTRACT

A process for the hydrogenation of a hydrocarbon feed containing unsaturated components includes providing a catalyst including at least one noble metal on a non-crystalline, mesoporous inorganic oxide support having at least 97 volume percent interconnected mesopores based upon mesopores and micropores, a BET surface area of at least 300 m²/g and a pore volume of at least 0.3 cm³/g; and, contacting the hydrocarbon feed with hydrogen in the presence of said catalyst under hydrogenation reaction conditions.

### CROSS REFERENCE TO RELATED APPLICATIONS

This application is a continuation-in-part of copending U.S. application Ser. No. 10/886,993 filed Jul. 8, 2004, which is herein incorporated by reference.

### BACKGROUND

1. Field of the Invention

The present invention relates to a process and catalyst for hydrogenating aromatics and olefins in hydrocarbon streams, preferably, but not limited to, hydrocarbon distillates.

2. Background of the Art

The removal of aromatics from various hydrocarbon distillates (e.g., jet fuel, diesel fuel, lube base stocks, etc.) can be difficult because of the wide variety of possible mixes of monocyclic and polycyclic aromatics. While dearomatization can require a considerable capital investment on the part of most refiners, it can also provide ancillary benefits. Distillate aromatics content is inextricably related to the cetane number, the primary measure of diesel fuel quality. The cetane number is highly dependent upon the paraffinicity and saturation of the hydrocarbon molecules, and whether they are straight chain molecules or have alkyl side chains attached to rings. A distillate stream comprising mostly aromatic molecules with few or no alkyl side chains is generally of lower cetane quality, whereas a highly paraffinic stream is generally of higher cetane quality. Jet fuel quality is also dependent upon lower aromatics content because of the aromatics/smoke point relationship. Most jet fuels are limited by specification to an aromatics content of 25 volume percent (max.).

An increased demand for more paraffinic distillates is also the result of the regulatory environment. Dearomatization has been of increasing importance because of government legislation that mandates substantial reductions in distillate aromatics and polynuclear aromatics content. The current U.S. Environmental Protection Agency specification for diesel fuel limits the aromatics content of diesel fuel to a maximum of 35 volume percent. The California diesel fuel specification is 10-volume percent maximum.

Many parts of the world are experiencing a phenomenon called “dieselization,” which refers to an upward shift of the diesel fuel/gasoline fuel demand ratio along with a general increase in the demand for fuel. Worldwide diesel fuel demand is projected to double between the years 2000 and 2010, partly in response to economic growth, efforts to combat global warming, and general demands for fuel efficiency. One approach to meet these demands will be to shift the use of lower quality home heating oil to automotive diesel fuel. This will result in the increased necessity of desulfurization and dearomatization.

However, the need for more paraffinic distillates leads to harsher reaction conditions for the conventional hydrogenation metal catalyst such as cobalt, molybdenum, nickel and tungsten. In recent years, the use of mixed noble metals on a support or zeolite has proven to yield a highly active dearomatization catalyst.

U.S. Pat. No. 5,151,172 to Kukes et al. discloses a process for the hydrogenation of distillate Feedstocks over a catalyst comprising a combination of palladium and platinum on a zeolite (i.e., mordenite) support.

U.S. Pat. No. 5,147,526 to Kukes et al. discloses a process for the hydrogenation of distillate feedstock over a catalyst comprising a combination of palladium and platinum on a support of zeolite Y with about 1.5 wt % to about 8.0 wt % sodium.

U.S. Pat. No. 5,346,612 to Kukes et al. disclose a process using a combination of palladium and platinum on a zeolite beta support.

U.S. Pat. No. 5,451,312 to Apelian et al. discloses platinum and palladium on a mesoporous, crystalline support, MCM-41. The use of the mesoporous support provides the advantage of reducing mass transfer limitations via a significantly larger pore system. However, although the mesoporous support provides better molecular access as compared with the zeolitic system, the crystalline mesoporous material is nevertheless limited because of the lack of interconnectivity of the pores. Furthermore, only a limited variation of the oxide used in the crystalline mesoporous support is possible without disturbing the crystalline structure of the support.

What is needed is a mesoporous catalyst system that provides a system of highly interconnected mesopores having pore sizes that are selectable within a wide range, and having greater flexibility in choosing the inorganic oxide components of the structure.

### SUMMARY OF THE INVENTION

A process for the hydrogenation of a hydrocarbon feed containing unsaturated components is provided herein. The process comprises providing a catalyst including at least one Group VIII metal on a noncrystalline, mesoporous inorganic oxide support having at least 97 volume percent interconnected mesopores based upon mesopores and micropores, a BET surface area of at least 300 m²/g and a pore volume of at least 0.3 cm³/g; and, contacting the hydrocarbon feed with hydrogen in the presence of said catalyst under hydrogenation reaction conditions.

The present invention provides a mesoporous catalyst system that provides a system of highly interconnected mesopores having pore sizes that are tunable within a wide range, and having greater flexibility in choosing the inorganic oxide components of the structure. Moreover, the system of the invention allows for the dispersion of a zeolite within the mesoporous matrix, which significantly enhances the access to the small crystal zeolite.

### DETAILED DESCRIPTION OF PREFERRED EMBODIMENT(S)

This invention provides a process for the saturation (hydrogenation) of a distillate hydrocarbon feedstock containing aromatics and/or olefins with a catalyst including one or more noble metals on a catalyst support that provides a reduction of the unsaturated components in the feedstock.

While other petroleum streams can benefit from this invention, the preferred distillate hydrocarbon feedstock processed in the present invention can be any refinery stream boiling in a range from about 150° F. (66° C.) to about 700° F. (371° C.), preferably 300° F. (149° C.) to about 700° F. (371° C.), and more preferably between about 350° F. (177° C.) and about 700° F. (371° C.).

A feature of the present invention is the ability to process hydrocarbon feeds having aromatic contents of over 20% by weight, over 50% by weight over 70% by weight, even up to 80% by weight.

The distillate hydrocarbon feedstock can comprise high and low sulfur virgin distillates derived from high- and low-sulfur crudes, coker distillates, catalytic cracker light and heavy catalytic cycle oils, visbreaker distillates and distillate boiling range products from hydrocracker, FCC or TCC feed hydrotreater and resid hydrotreater facilities. Generally, the light and heavy catalytic cycle oils are the most highly aromatic feedstock components, ranging as high as 80% by weight (FIA). The majority of cycle oil aromatics are present as monoaromatics and di-aromatics with a smaller portion present as tri-aromatics.

Virgin stocks such as high and low sulfur virgin distillates are lower in aromatics content ranging as high as 20% by weight aromatics (FIA). Generally, the aromatics content of a combined hydrogenation facility feedstock will range from about 5% by weight to about 80% by weight, more typically from about 10% by weight to about 70% by weight, and most typically from about 20% by weight to about 60% by weight. In a distillate hydrogenation facility it is generally more profitable to process feedstocks in order of highest aromaticity since catalytic processes often proceed to equilibrium product aromatics concentrations at sufficiently low space velocity.

The distillate hydrocarbon feedstock sulfur concentration is generally a function of the high and low sulfur crude mix, the hydroprocessing capability of a refinery per barrel of crude capacity, and the alternative dispositions of distillate feedstock components. The higher sulfur distillate feedstock components are generally coker distillate, visbreaker distillates, and catalytic cycle oils. These distillate feedstock components can have total nitrogen concentrations ranging as high as 2,000 ppm, but generally range from about 5 ppm to about 900 ppm.

Particularly preferred feedstocks for the present invention are hydrocarbon fractions in the jet fuel and diesel fuel boiling range of 150-400° C. Typical aromatic compounds contained in the feedstocks include mono-aromatic, di-aromatic, and tri-aromatics, particularly those normally boiling below about 343° C. Examples of aromatics contained in the feedstocks include mono-aromatics such as alkyl benzenes, indans/tetralins and dinaphthene benzenes, di-aromatics such as naphthalenes, biphenyls and fluorenes, and tri-aromatics such as phenanthrenes and naphphenanthrenes. Although feedstocks containing a substantial proportion of poly-aromatics are preferred (i.e., up to 100 weight percent of the total aromatics in such feedstocks can be comprised of poly-aromatics), a commonly processed feedstock of the invention contains a substantial proportion of mono-aromatics and a relatively small proportion of polyaromatics. The mono-aromatic content of the total aromatics in the feedstock is usually greater than 50 weight percent. For use herein, typical hydrocarbon distillate fractions, or mixtures thereof, contain at least about 10 volume percent of aromatic hydrocarbon compounds. The most highly preferred feedstock process in the present invention is a diesel fuel feedstock containing at least 10, often at least 20, and commonly more than 30 volume percent of aromatic containing compounds, with typical ranges from about 10 to about 80 and often about 20 to 50 volume percent. The maximum benefit of the process of the present invention is achieved as higher concentrations of the aromatics in the feedstock are saturated without substantial cracking of homocyclic aromatics.

Another preferred feedstock encompasses hydrocarbons of lubricant viscosity. The upgrading process may be carried out with mineral oil lubricants or synthetic hydrocarbon lubricants, of which the poly alpha-olefins (“PAO”) materials are exemplified, both conventional type PAOs prepared using Friedel-Crafts type catalysts as well as the HVI-PAO materials produced using a reduced Group VIB (Cr, Mo, W) metal oxide catalyst.

The mineral oil lubricants may generally be characterized as having a minimum boiling point of at least 650° F. (343° C.); and usually they will be neutral, i.e., distillate, stocks with a 95% boiling point of not more than 1050° F. (566° C.) although residual lube stocks, such as bright stock, may also be treated by the same catalytic process. Mineral oil stocks of this kind have historically been prepared by the conventional refining process involving atmospheric and vacuum distillation of a crude of suitable composition, followed by removal of undesirable aromatic components via solvent extraction using a solvent such as phenol, furfural or N,N-dimethylformamide (“DMF”). Dewaxing to the desired product pour point may be carried out using either solvent dewaxing or catalytic dewaxing techniques (or a combination thereof), and it is particularly preferred that a hydrogenative treatment according to the present invention should follow any catalytic dewaxing treatment in order to saturate lube boiling range olefins which may be produced during the catalytic dewaxing process.

Mineral oil stocks can also be prepared by catalytic hydrocracking, wherein the unconverted, high boiling hydrocarbon stream serves as the waxy lube base. Subsequent to the hydrocracking process, the lube stock is then subjected to dewaxing and hydrofinishing to adjust fluidity and reduce olefins and possibly aromatics. This process, commonly called “lube hydrocracking”, is often employed when the feedstock is inadequate for conventional lube processing or when a high VI lube product is required.

The present process is also applicable to the hydrogenative treatment of synthetic lubricating oils, especially the poly alpha-olefins (“PAOs”) including the HVI-PAO type materials. These types of lubricants may be produced by polymerization or oligomerization using Friedel-Crafts type catalysts such as aluminum trichloride, boron trifluoride or boron trifluoride complexes, e.g., with water, lower alkanols or esters in the conventional manner. The HVI-PAO type oligomers may be prepared by the methods described in U.S. Pat. No. 4,827,064 or 4,827,073, using a reduced Group VIB metal oxide catalyst, normally chromium on silica. The HVI-PAO materials include the higher molecular weight versions prepared by the use of lower oligomerization temperatures, as disclosed in U.S. Pat. No. 5,012,020. The HVI-PAO materials are characterized by a branch ratio below 0.19 which results from the use of the unique reduced metal oxide catalyst during the oligomerization process.

The lubricant materials are subjected to the hydrogenative treatment in the presence of a catalyst that comprises a metal component for hydrogenation together with the inventive mesoporous material and, optionally, a binder.

The hydrogenation reaction is carried out under conventional conditions with temperatures from about 100° to about 700° F. and preferably in the range of 150° to 500° F. The hydrogen is preferably under superatmospheric conditions and hydrogen partial pressure may vary up to about 2,500 psi but normally will be from about 100 to 1500 psi. Hydrogen circulation rates are typically in excess of that required stochiometrically for complete saturation ranging from 200% to 5000% stochiometric excess. Once-through circulation is preferred in order to maximize the purity of the hydrogen. Space velocities are typically in the range of 0.1 to 10 LHSV, usually from 1 to 3 LHSV. The products of the hydrogenation reaction have a low degree of unsaturation consistent with the hydrogenative treatment. In most cases hydrocarbon lubricant feeds having a bromine number greater than 5 can be processed according to the method of the invention to provide a product having a bromine number less than 3, and often less than 1.

Where the particular hydroprocessing facility is a two-stage process, the first stage is often designed to desulfurize and denitrogenate, and the second stage is designed to dearomatize. In these operations, the feedstocks entering the dearomatization stage are substantially lower in nitrogen and sulfur content and can be lower in aromatics content than the feedstocks entering the hydroprocessing facility.

The hydrogenation process of the present invention generally begins with a distillate feedstock-preheating step. The feedstock is preheated in feed/effluent heat exchangers prior to entering a furnace for final preheating to a targeted reaction zone inlet temperature. The feedstock can be contacted with a hydrogen stream prior to, during, and/or after preheating. The hydrogen-containing stream can also be added in the hydrogenation reaction zone of a single-stage hydrogenation process or in either the first or second stage of a two-stage hydrogenation process.

The hydrogen stream can be pure hydrogen or can be in admixture with diluents such as hydrocarbon, carbon monoxide, carbon dioxide, nitrogen, water, sulfur compounds, and the like. The hydrogen stream purity should be at least about 50% by volume hydrogen, preferably at least about 65% by volume hydrogen, and more preferably at least about 75% by volume hydrogen for best results. Hydrogen can be supplied from a hydrogen plant, a catalytic reforming facility, or other hydrogen-producing processes.

The reaction zone can consist of one or more fixed bed reactors containing the same or different catalysts. Two-stage processes can be designed with at least one fixed bed reactor for desulfurization and denitrogenation, and at least one fixed bed reactor for dearomatization. A fixed bed reactor often comprises a plurality of catalyst beds. Optionally, the effluent of one fixed bed can be cooled before it is directed into a subsequent fixed bed. The plurality of catalyst beds in a single fixed bed reactor can also comprise the same or different catalysts. Where the catalysts are different in a multi-bed fixed bed reactor, the initial bed or beds are generally for desulfurization and denitrogenation, and subsequent beds are for dearomatization. When a multi-reactor system is employed, the interreactor gas undergoes a hot “strip” to remove H₂S and NH₃. These first-stage product gases can cause reaction inhibition and, more importantly, can poison the noble metal(s) on the dearomatization catalysts.

Since the hydrogenation reaction is generally exothermic, interstage cooling, via hydrogen injection can be employed. Other methods, including interstage heat transfer, can be employed. Two-stage processes can provide reduced temperature exotherms per reactor shell and provide better overall reactor temperature control, important for safety and optimal catalyst efficiency and longevity

The reaction zone effluent is generally cooled, and the effluent stream is directed to a separator device to remove the hydrogen. One example of this is an amine scrubber. The H₂S is sent to the sulfur recovery unit, and the NH₃ is often collected as a refinery byproduct. Some of the recovered hydrogen can be recycled back to the process while some of the hydrogen can be cascades to other, less demanding hydroprocessing units (e.g., naphtha pretreaters), or purged to external systems such as plant or refinery fuel. The hydrogen purge rate is often controlled to maintain a minimum hydrogen purity and remove hydrogen sulfide. Recycled hydrogen is generally compressed, supplemented with “make-up” hydrogen, and reinjected into the process for further hydrogenation. One preferred disposition strategy of the low purity hydrogen is to go back to the hydrogen plant loop, where an absorber recovers much of the hydrogen upstream of the hydrogen unit.

The separator device liquid effluent can then be processed in a stripper device where light hydrocarbons can be removed and directed to more appropriate hydrocarbon pools. The stripper liquid effluent product is then generally conveyed to blending facilities for production of finished distillate products.

Operating conditions to be used in the hydroprocessing step of the present invention include an average reaction zone temperature of from about 300° F. (150° C.) to about 750° F. (400° C.), preferably from about 500° F. (260° C.) to about 650° F. (343° C.), and most preferably from about 525° F. (275° C.) to about 625° F. (330° C.) for best results. Reaction temperatures below these ranges can result in less effective hydrogenation. Excessively high temperatures can cause the process to reach a thermodynamic aromatic reduction limit, non-selective hydrocracking, catalyst deactivation, and increase energy costs.

The process of the present invention generally operates at reaction zone hydrogen partial pressures ranging from about 200 psi to about 2,000 psi, more preferably from about 500 psi to about 1,500 psi, and most preferably from about 600 psi to about 1,200 psi for best results. Hydrogen circulation rates generally range from about 500 SCF/Bbl to about 20,000 SCF/Bbl, preferably from about 2,000 SCF/Bbl to about 15,000 SCF/Bbl, and most preferably from about 3,000 to about 13,000 SCF/Bbl for best results. Reaction pressures and hydrogen circulation rates below these ranges can result in higher catalyst deactivation rates as well as in less effective desulfurization, denitrogenation, and dearomatization. Excessively high reaction pressures increase energy and equipment costs and provide diminishing marginal benefits.

The process of the present invention generally operates at a liquid hourly space velocity of from about 0.2 hr⁻¹ to about 10.0 hr⁻¹, preferably from about 0.5 hr⁻¹ to about 3.0 hr⁻¹, and most preferably from about 1.0 hr⁻¹ to about 2.0 hr⁻¹ for best results. Excessively high space velocities can result in reduced overall hydrogenation.

The catalyst support, denoted as TUD-1, is a three-dimensional noncrystalline, mesoporous inorganic oxide material containing at least 97 volume percent interconnected mesopores (i.e., no more than 3 volume percent micropores) based on micropores and mesopores of the organic oxide material, and generally at least 98 volume percent mesopores. A method for making a preferred porous catalyst support is described in U.S. Pat. No. 6,358,486 and U.S. patent application Ser. No. 10/764,797 filed Jan. 26, 2004 (“Method For Making Mesoporous or Combined Mesoporous and Microporous Inorganic Oxides”), both of which are herein incorporated by reference. The average mesopore size of the preferred catalyst as determined from N₂-porosimetry ranges from about 2 nm to about 25 nm. Generally, the mesoporous inorganic oxide is prepared by heating a mixture of (1) a precursor of the inorganic oxide in water, and (2) an organic pore-forming agent at a certain temperature for a certain period of time.

The starting material is generally an amorphous material and may be comprised of one or more inorganic oxides such as silicon oxide or aluminum oxide, with or without additional metal oxides. The silicon atoms may be replaced in part by metal atoms such as aluminum, titanium, vanadium, zirconium, gallium, manganese, zinc, chromium, molybdenum, nickel, cobalt and iron and the like. Preferably, the inorganic oxide is selected from the group consisting of silica, alumina, silica-alumina, titania, zirconia, magnesia, and combinations thereof. The additional metals may optionally be incorporated into the material prior to initiating the process for producing a structure that contains mesopores. Also, after preparation of the material, cations in the system may optionally be replaced with other ions such as those of an alkali metal (e.g., sodium, potassium, lithium, etc.). The alkali cations can titrate any residual acidity that is present in the TUD-1, especially when in the Al-TUD-1 or Al—Si-TUD-1 form. Residual acidity can cause unwanted cracking reactions and thereby lower overall, liquid product yield.

The mesoporous catalyst support is a noncrystalline material (i.e., no crystallinity is observed by presently available X-ray diffraction techniques). The d spacing of the mesopores is preferably from about 3 nm to about 30 nm. The surface area of the catalyst support as determined by BET (N₂) is at least about 300 m²/g and preferably ranges from about 400 m²/g to about 1200 m²/g. The catalyst pore volume is at least about 0.3 cm³/g and preferably ranges from about 0.4 cm³/g to about 2.2 cm³/g.

There are many ways to prepare the catalyst support, TUD-1, but these ways can be classified into two types depending on the starting materials of inorganic oxides: (1) organic-containing precursors, and (2) inorganic precursors. In the first case the inorganic oxide precursor can preferably be an alkoxide having desired elements selected from silicon, aluminum, titanium, vanadium, zirconium, gallium, manganese, zinc, chromium, molybdenum, nickel, cobalt and iron, for example, an organic silicate such as tetraethyl orthosilicate (TEOS), or an organic source of aluminum oxide such as aluminum isopropoxide. TEOS and aluminum isopropoxide are commercially available from known suppliers.

The pH of the solution is preferably kept above 7.0. Optionally, the aqueous solution can contain other metal ions such as those indicated above. After stirring, an organic mesopore-forming agent which binds to the silica (or other inorganic oxide) species by hydrogen bonding is added and mixed into the aqueous solution. The organic pore-forming agent is preferably a glycol (a compound that includes two or more hydroxyl groups), such as glycerol, diethylene glycol, triethylene glycol, tetraethylene glycol, propylene glycol, and the like, or member(s) of the group consisting of triethanolamine, sulfolane, tetraethylene pentamine and diethylglycol dibenzoate. The organic pore-forming agent should not be so hydrophobic so as to form a separate phase in the aqueous solution, and is preferably added by dropwise addition with stirring to the aqueous inorganic oxide solution. After a period of time (e.g., from about 1 to 2 hours) the mixture forms a thick gel. The mixture is preferably stirred during this period of time to facilitate the mixing of the components. The mixture preferably includes an alkanol, which can be added to the mixture and/or formed in-situ by the decomposition of the inorganic oxide precursor. For example, TEOS, upon heating, produces ethanol. Propanol may be produced by the decomposition of aluminum isopropoxide.

The second type of synthesis route to get the same gel is the use of inorganic precursors as starting materials. The preferred inorganic precursors comprise of oxides and/or hydroxide oxides having desired elements selected from silicon, aluminum, titanium, vanadium, zirconium, gallium, manganese, zinc, chromium, molybdenum, nickel, cobalt and iron. The precursor is first mixed with one or more pore-forming agents and heated up to 120-250° C. for a certain period of time, e.g. 2-10 hours, sufficient to convert the inorganic precursor into organic-containing complexes. The complexes then are mixed with water to hydrolyze and obtain a homogenous thick gel.

The gel obtained by two types of methods described above is then aged at a temperature of from about 5° C. to about 45° C., preferably at room temperature, to complete the hydrolysis and poly-condensation of the inorganic oxide source. Aging preferably can take place for up to about 48 hours, generally from about 2 hours to 30 hours, more preferably from about 10 hours to 20 hours. After the aging step the gel is heated in air at about 90° C. to 100° C. for a period of time sufficient to dry the gel by driving off water (e.g., from about 6 to about 24 hours). Preferably, the organic pore-forming agent, which helps form the mesopores, should remain in the gel during the drying stage. Accordingly, the preferred organic pore-forming agent has a boiling point of at least about 150° C.

The dried material, which still contains the organic pore-forming agent, is heated to a temperature at which there is a substantial formation of mesopores. The pore-forming step is conducted at a temperature above the boiling point of water and up to about the boiling point of the organic pore-forming agent. Generally, the mesopore formation is carried out at a temperature of from about 100° C. to about 250° C., preferably from about 150° C. to about 200° C. The pore-forming step can optionally be performed hydrothermally in a sealed vessel at autogenous pressure. The size of the mesopores and volume of the mesopores in the final product are influenced by the duration and temperature of the hydrothermal step. Generally, increasing the temperature and duration of the treatment increases the percentage of mesopore volume in the final product.

After the pore-forming step the catalyst material is calcined at a temperature of from about 300° C. to about 1000° C., preferably from about 400° C. to about 700° C., more preferably from about 500° C. to about 600° C., and maintained at the calcining temperature for a period of time sufficient to effect calcination of the material. The duration of the calcining step typically ranges from about 2 hours to about 40 hours, preferably 5 hours to 15 hours, depending, in part, upon the calcining temperature.

To prevent hot spots the temperature should be raised gradually. Preferably, the temperature of the catalyst material should be raised to the calcining temperature at a rate of from about 0.1° C./min. to about 25° C./min., more preferably from about 0.5° C./min. to about 15° C./min., and most preferably from about 1° C./min. to about 5° C./min.

During calcining the structure of the catalyst material is finally formed while the organic molecules are expelled from the material and decomposed.

The calcination process to remove organic pore-forming agent can be replaced by extraction using organic solvents, e.g., ethanol. In this case the pore-forming agent can be recovered for reuse.

Also, the catalyst powder of the present invention can be admixed with binders such as silica and/or alumina, and then formed into desired shapes (e.g., pellets, rings, etc.) by extrusion or other suitable methods.

The catalyst includes at least one metal component selected from Group VIII of the Periodic Table of the Elements, which includes iron, cobalt, nickel, and the noble metals, i.e., platinum, palladium, ruthenium, rhodium, osmium and iridium. Especially preferred metals include platinum, palladium, rhodium, iridium and nickel. The amount of Group VIII metal is at least about 0.1 wt. % based upon the total catalyst weight

The Group VIII metal can be incorporated into the inorganic mesoporous oxide by any suitable method such as ion exchange or by impregnating the inorganic oxide with a solution of a soluble, decomposable compound of the Group VIII metal, then washing, drying, and subjecting the impregnated inorganic oxide to a process such as calcining to decompose the Group VIII metal compound, thereby producing an activated catalyst having free Group VIII metal in the pores of the inorganic oxide. Suitable Group VIII metal compounds include salts such as nitrates, chlorides, ammonium complexes, and the like.

Washing of the Group VIII metal impregnated inorganic oxide catalyst is optionally performed with water to remove some anions. Drying of the catalyst to remove water and/or other volatile compounds can be accomplished by heating the catalyst to a drying temperature of from about 50° C. to about 190° C. Calcining to activate the catalyst can be performed at a temperature of from about 150° C. to about 600° C. for a sufficient period of time. Generally, calcining can be performed for 2 to 40 hours depending, at least in part, on the calcining temperature.

Optionally, one or more zeolite can be incorporated into the catalyst and dispersed throughout the mesoporous matrix. The zeolite is preferably added to the inorganic oxide precursor-water solution prior to the formation of the mesoporous structure. Suitable zeolites include, for example, FAU, EMT, BEA, VFI, AET and/or CLO. The zeolite is preferably present in an amount of 0.05 wt. % to 50 wt. %, based on the total catalyst weight.

Another preferred type of hydrogenation encompasses the selective removal of impurities in a feed containing hydrocarbons. More particularly, it relates to the process of selective hydrogenation of compounds containing a triple bond and/or compounds having two or more double bonds as opposed to a compound having a single double bond and the selective hydrogenation of compounds having two adjacent double bonds as opposed to those where the two double bonds are separated by one or more single bonds.

Such reactions include, but are not limited to, the selective hydrogenation of acetylenic and/or dienic impurities in a feed containing at least one monoolefin. Further examples are the selective hydrogenation of acetylene in an ethylene stream, the selective hydrogenation of methylacetylene and propadiene in a propylene stream, the selective hydrogenation of butadiene in a butene stream, and the selective hydrogenation of vinyl and ethyl acetylene, and 1,2-butadiene in a feed containing 1,3-butadiene.

In the petrochemical industry, produced streams contain one or more monoolefins, and as impurities contain acetylenic and/or dienic compounds. Acetylenic impurities include acetylene, methylacetylene and diacetylene. Dienic impurities include 1,2-butadiene, 1,3-butadiene, and propadiene.

Such a stream is usually subjected to selective hydrogenation to minimize/remove the acetylenic and/or dienic impurities without hydrogenating the desired monoolefins. Such a process may be accomplished by catalytic selective hydrogenation, using a catalyst.

This catalyst comprises a metal, preferably a noble metal, supported on the inventive mesoporous material and optionally, a binder. This catalyst may also contain additional metals used as promoters.

The selective hydrogenation of the acetylenic and/or dienic impurities is carried out in a single stage hydrogenation in the presence of the catalyst described hereinabove. The feed is introduced as a liquid and may be partially or completely vaporized during the hydrogenation. The feed to be selectively hydrogenated and stream of hydrogen gas are introduced into the reactor, at a temperature from about 0° C. to 50° C. The reactor is operated in the pressure range of 200 psi to 500 psi. Depending on the level of acetylenic and/or dienic impurities in the feed, the inlet temperature, and the allowable outlet temperature, it may be necessary to recycle a portion of the product to the reaction zone.

The amount of hydrogen that is introduced into the reactor is based on the amount of the impurities in the feed. Hydrogen may be introduced into the reactor with a suitable diluent, such as methane.

A suitable liquid hourly space velocity should be used and should be apparent to those skilled in the art.

The following examples illustrate features of the invention.

### EXAMPLE 1

This example demonstrates a synthesis process of Si-TUD-1 using silicon alkoxides as silica source. 736 parts by weight of tetraethyl orthosilicate (98%, ACROS) was mixed with 540 parts of triethanolamine (TEA) (97%, ACROS) while stirring. After half an hour, 590 parts of water were added slowly into the above mixture while stirring. After another half an hour, 145 parts of tetraethylammonium hydroxide (TEOH) (35 wt %) was added into the above mixture to obtain a homogeneous gel. The gel was aged at room temperature for 24 hr. Next, the gel was dried at about 98° C. for 18 hr, and calcined at 600° C. in air for 10 hr. with a heating rate of 1° C./min.

The X-ray diffraction (XRD) pattern of the final material showed an intensive 2Θ peak of <2°, indicating a mesoporous structure. BET measurement using nitrogen adsorption revealed a surface area of 683 m²/g, average pore diameter of about 4.0 mm and total pore volume of about 0.7 cm³/g.

### EXAMPLE 2

This example demonstrates a synthesis process of Si-TUD-1 using silica gel as silica source. First, 24 parts of silica gel, 76 parts of TEA and 62 parts of ethylene glycol (EG) were loaded into a reactor equipped with a condenser. After the contents of the reactor were mixed well with a mechanical stirrer, the mixture was heated up to 200-210° C. while stirring. This setup removed most of water generated during reaction together with a small portion of EG from the top of the condenser. Meanwhile, most of the EG and TEA remained in the reaction mixture. After about 8 hours, heating was stopped; and a slightly brown, glue-like complex liquid was collected after cooling down to room temperature.

Second, 100 parts of water were added into 125 parts of the complex liquid obtained above under stirring conditions. After one hour stirring, the mixture formed a thick gel; the gel was aged at room temperature for 2 days.

Third, the thick gel was dried at 98° C. for 23 hours, and then loaded into autoclave and heated up to 180° C. for 6 hours. Finally, it was calcined at 600° C. in air for 10 hours with a heating rate of 1° C./min.

The X-ray diffraction (XRD) pattern of the final material showed an intensive 2Θ peak of <2°, indicating a mesoporous structure. BET measurement using nitrogen adsorption revealed a surface area of 556 m²/g, average pore diameter of about 8.1 nm and total pore volume of about 0.92 cm³/g.

### EXAMPLE 3

This example illustrates Al—Si-TUD-1 synthesis. First, 250 parts of silica gel, 697 parts of TEA and 287 parts of ethylene glycol (EG) were loaded into a reactor equipped with a condenser. After the contents of the reactor were mixed well with a mechanical stirrer, the mixture was heated up to 200-210° C. while stirring. This arrangement removed most of the water generated during the reaction together with a small portion of EG from the top of the condenser. Meanwhile, most of the EG and TEA remained in the reaction mixture. After about 3 hours, the reactor was cooled down to 100° C.; and to the reactor was added another mixture comprising 237 parts of aluminum hydroxide, 207 g EG and 500 g TEA. The mixture was heated up again to 200-210° C., and after 4 hours heating was stopped. A slightly brown, glue-like complex liquid was collected after cooling the mixture down to room temperature.

Second, 760 parts of water and 350 parts of tetraethylammonium hydroxide were added into the complex liquid obtained above under stirring conditions. After one hour stirring, the mixture formed a thick gel; the gel was aged at room temperature for 1 day.

Third, the thick gel was dried at 98° C. for 23 hours, and then loaded into autoclave and heated up to 180° C. for 16 hours. Finally, it was calcined at 600° C. in air for 10 hours with a heating rate of 1° C./min.

The X-ray diffraction (XRD) pattern of the final material showed an intensive 2Θ peak of <2°, indicating a mesoporous structure. BET measurement using nitrogen adsorption revealed a surface area of 606 m²/g, average pore diameter of about 6.0 nm and total pore volume of about 0.78 cm³/g.

### EXAMPLE 4

This example demonstrates catalyst preparation of 0.90 wt % iridium/Si-TUD-1 by incipient wetness. 0.134 Parts of iridium (III) chloride were dissolved in 5.2 parts of deionized water. This solution was added to 8 parts of Si-TUD-1 obtained in Example 1 with mixing. The powder was dried at 25° C.

For dispersion measurement using CO chemisorption, the powder was then reduced in a hydrogen stream at 100° C. for 1 hr. followed by heating to 350° C. at 5° C./min. and was maintained at this temperature for 2 hr. CO chemisorption showed a 75% dispersion for the metal assuming an Ir:CO stoichiometry of 1.

### EXAMPLE 5

This example demonstrates the preparation of 0.9 wt % palladium and 0.3 wt % platinum/Si-TUD-1 by incipient wetness. Al—Si-TUD-1 obtained in Example 3 was first extruded. Then 70 parts of 1/16″ extrudates were impregnated with an aqueous solution comprising 0.42 parts of tetraammine platinum nitrate, 12.5 parts of aqueous solution of tetraammine palladium nitrate (5% Pd) and 43 parts of water. Impregnated Al—Si-TUD-1 was aged at room temperature for 6 hours before dried at 90° C. for 2 hours. The dried material was finally calcined in air at 350° C. for 4 hours with a heating rate of 1° C./min. Noble metal dispersion was measured using CO chemisorption; the powder was then reduced in a hydrogen stream at 100° C. for 1 hr. followed by heating to 350° C. at 5° C./min. and was maintained at this temperature for 2 hr. A dispersion of 51% was measured for the metal assuming a Pt:CO stoichiometry of 1.

### EXAMPLE 6

This example demonstrates the preparation of 0.46 wt % platinum/Si-TUD-1 by incipient wetness. 0.046 Parts of tetraammine platinum (II) nitrate were dissolved in 4.1 parts of deionized water. This solution was added to 5 parts of Si-TUD-1 obtained in Example 1 with mixing. The powder was dried at 25° C.

For dispersion measurement using CO chemisorption, the powder was then reduced in a hydrogen stream at 100° C. for 1 hr. followed by heating to 350° C. at 5° C./min. and was maintained at this temperature for 2 hr. A dispersion of 72% was measured for the sample assuming a Pt:CO stoichiometry of 1.

### EXAMPLE 7

21 Parts of Si-TUD-1 obtained in Example 1 were suspended in deionized water. The pH of the solution was adjusted to 2.5 by adding nitric acid. The exchange was carried out for 5 hr. The solution was then drained. The Si-TUD-1 was then washed 5 times with deionized water. This Si-TUD-1 was then placed in 600 parts of deionized water. The pH of this solution was adjusted to 9.5 using ammonium nitrate. This exchange was carried out for 1 hr. During this exchange, ammonium nitrate was added as needed to maintain the pH at 9.5. After the exchange, the Si-TUD-1 was washed 5 times with deionized water. Si-TUD-1 was then dried at 25° C. A 0.50% palladium/Si-TUD-1 was prepared utilizing this acid/base-treated Si-TUD-1, from an incipient wetness of tetraammine palladium (ii) nitrate. 0.071 Parts of the palladium salt were dissolved in 4.1 parts of deionized water. This solution was added to 5 parts of the Si-TUD-1 with mixing. The powder was dried at 25° C. The catalyst powder was then calcined in air at 350° C. for 2 hr, using a heating rate of 1° C./min.

For dispersion measurement using CO chemisorption, the calcined powder was then reduced in a hydrogen stream at 100° C. for 1 hr. followed by heating to 350° C. at 5° C./min and was maintained at this temperature for 2 hr. A dispersion of 96% was measured for the sample assuming a Pd:CO stoichiometry of 1.

### EXAMPLE 8

This example demonstrates the preparation of 0.25% palladium/Si-TUD-1 utilizing the acid/base-treated TUD-1 (Example 7), from an incipient wetness of tetraammine palladium (II) nitrate. 0.035 Parts of the palladium salt were dissolved in 3.9 parts of deionized water. This solution was added to 5 parts of the Si-TUD-1 with mixing. The powder was dried at 25° C. The catalyst powder was then calcined in air at 350° C. for 2 hr., using a heating rate of 1° C./min.

For dispersion measurement using CO chemisorption, the calcined powder was then reduced in a hydrogen stream at 100° C. for 1 hr, followed by heating to 350° C. at 5° C./min, and was maintained at this temperature for 2 hr. A dispersion of 90% was measured for the sample assuming a Pd:CO stoichiometry of 1.

### EXAMPLE 9

A 0.38 wt % palladium/0.23 wt % platinum/Si-TUD-1 catalyst was prepared as follows. A 0.38% palladium TUD-1 was prepared utilizing the acid/base-treated Si-TUD-1 (Example 7), from an incipient wetness of tetraammine palladium (II) nitrate. 0.053 Parts of the palladium salt were dissolved in 3.75 parts of deionized water. This solution was added to 5 parts of the Si-TUD-1 with mixing. The powder was dried at 25° C. The catalyst powder was then calcined in air at 350° C. for 2 hr. using a heating rate of 1° C./min.

A 0.23 wt % platinum impregnation on this catalyst was prepared from an incipient wetness of tetraammine platinum (II) nitrate. 0.018 Parts of the platinum salt were dissolved in 3.25 parts of deionized water. This solution was added to 4.02 parts of 0.38 wt % Pd/Si-TUD-1 with mixing. The powder was dried at 25° C.

For dispersion measurement using CO chemisorption, the powder was then reduced in a hydrogen stream at 100° C. for 1 hr. followed by heating to 350° C. at 5° C./min and was maintained at this temperature for 2 hr. A dispersion of 81% was measured for the sample assuming Pd:CO and Pt:CO stoichiometry of 1.

### EXAMPLE 10

A 0.19 wt % palladium/0.11 wt % platinum/Si-TUD-1 catalyst was prepared as follows. A 0.19 wt % palladium/Si-TUD-1 was prepared utilizing the acid/base-treated Si-TUD-1 (Example 7), from an incipient wetness of tetraammine palladium (II) nitrate. 0.027 parts of the palladium salt was dissolved in 3.5 parts of deionized water. This solution was added to 5 parts of Si-TUD-1 with mixing. The powder was dried at 25° C. The catalyst powder was then calcined in air at 350° C. for 2 hr. using a heating rate of 1° C./min.

A 0.11 wt % platinum impregnation on this catalyst was prepared from an incipient wetness of tetraamine platinum (ii) nitrate. 0.009 Parts of the platinum salt were dissolved in 3.27 parts of deionized water. This solution was added to 4.05 parts of 0.19% Pd/Si-TUD-1 with mixing. The powder was dried at 25° C.

For dispersion measurement using CO chemisorption, the powder was then reduced in a hydrogen stream at 100° C. for 1 hr. followed by heating to 350° C. at 5° C./min and was maintained at this temperature for 2 hr. A dispersion of 54% was measured for the sample assuming Pd:CO and Pt:CO stoichiometry of 1.

### EXAMPLE 11

Catalysts of TUD-1 were evaluated in a 1″ reactor with continuous real feed and compared with commercial catalyst. Table 1 summarizes the operation conditions. Table 2 shows the properties of the feed and the effluents, yield of the final products. It is clear that TUD-1 catalyst gave a final product having only 5% aromatics, whereas the commercial catalyst generated a product containing 10% aromatics under high space velocity. TUD-1 catalyst showed higher activity of aromatic saturation.

TABLE 1
Aromatic saturation operation conditions

Catalyst

| | Commercial | TUD-1 catalyst |
|---|---|---|
| Hours on stream, hr. | 264 | 288 |
| Inlet temp. ° F. | 435 | 437 |
| Outlet temp. ° F. | 460 | 484 |
| Temperature rise, ° F. | 25 | 47 |
| Total pressure, psig | 725 | 725 |
| Overall LHSV, hour⁻¹ | 2.4 | 2.4 |
| Overall hydrogen rate, SCF/BBL | 1200 | 1200 |
| Carbon balance, wt. % recovery | 100 | 100 |

TABLE 2
Comparison of overall performance of TUD-1
catalyst and commercial catalyst

| Overall effluent | | Commercial | TUD-1 |
|---|---|---|---|
| properties | (Feed) | catalyst | catalyst |
| API gravity | (38.1) | 40.2 | 40.6 |
| Density @ 60 F., g/cc | (.8344) | 0.8241 | 0.8220 |
| Carbon, wt. % | (86.78) | 85.92 | 85.65 |
| Hydrogen, wt. % | (13.22) | 14.08 | 14.35 |
| Sulfur, ppm | (3) | 1 | 1 |
| Nitrogen, ppm | (1) | <1 | <1 |
| Refractive index @ 25° C. | (1.4607) | 1.4517 | 1.4498 |
| Fia saturates, vol % | (77.6) | 89.0 | 94.2 |
| Fia olefins, vol % | (1.2) | 0.9 | 0.7 |
| Fia aromatics, vol % | (21.2) | 10.1 | 5.1 |
| Cetane index (ASTM D976) | (44.7) | 46.7 | 47.8 |
| Cetane index (ASTM D4737) | (44.7) | 47.1 | 48.3 |
| Final product yield, wt % | | | |
| C5-180° F. | (0.0) | 0.01 | 0.01 |
| 180-350° F. | (9.6) | 11.11 | 10.84 |
| 350-500° F. | (54.9) | 58.58 | 57.75 |
| 500-550° F. | (17.8) | 16.87 | 17.53 |
| 550EF + | (17.7) | 14.44 | 15.20 |
| Total | (100) | 101.00 | 101.32 |
| Others | | | |
| C5 + yield, | | 102.25 | 102.84 |
| volume % of feed | | | |
| % desulfurization | | 66.3 | 66.2 |
| % denitrogenation | | 100.0 | 100.0 |
| Overall H cons., scf/bbl | | 560 | 730 |

### EXAMPLE 12

An aluminum-based TUD-1 was prepared in this example. Sixty-five (65) parts by weight of isopropanol and 85 parts of ethanol were added to a vessel with 53 parts of aluminum isopropoxide. After stirring at 50° C. for about 4 hours, 50 parts of tetraethylene glycol (TEG) were added drop-wise while stirring. After stirring for another 4 hours, 10 parts of water together with 20 parts of isopropanol and 18 parts of ethanol were added under stirring. After half an hour of stirring, the mixture became a white suspension, which was then aged at room temperature for 48 hours, and then dried in air at 70° C. for 20 hours, to obtain a solid gel. This solid gel was heated in an autoclave at 160° C. for 2.5 hours and finally calcined at 600° C. for 6 hours in air to produce mesoporous aluminum oxide.

The XRD pattern of the resulting calcined mesoporous aluminum oxide. There was intensive 2Θ peak at 1.6°, characteristic of meso-structured materials. N₂ porosimetry showed the pore size distribution to be narrowly centered around 4.6 nm. ²⁷Al NMR spectroscopic measurements showed three peaks corresponding to four-, five- and six-coordinated aluminum at 75, 35 and 0 ppm, respectively. In summary, this was a typical mesoporous material of the present invention with four-, five- and six-coordinated aluminum.

### EXAMPLE 13

This example demonstrates the use of this invention composition as a catalyst support for hydrogenation. First, 3.13 parts of the Al-TUD-1 from Example 12 (“Sample 12”) is impregnated with 2 parts of a solution of 3.1 wt.-% Pt(NH₃)₄(NO₃)₂ in water by the incipient wetness method. After drying and calcination in air at 350° C. for 2 hours, 50 parts of impregnated Sample 12 is filled in to the reactor, then reduced with hydrogen at 300° C. for 2 hours.

As a probe reaction, mesitylene hydrogenation is carried out in a fixed-bed reactor under a total pressure of 6 bars and having a feed with a mesitylene concentration of 2.2 mol % in hydrogen. In order to measure the catalyst's rate constant, the reaction temperature is varied in the range of 100 to 130° C. in 10° C. increments. The modified contact time based on the mass of catalyst is kept constant at 0.6 g_{cat.}*min*l⁻¹. The first order reaction rate constants based on the catalyst mass is 0.15 g_{cat.}⁻¹*min⁻¹*l at 100° C.

### EXAMPLE 14

This example illustrates the selective hydrogenation of acetylenes and dienes. A Pd—Ag Al-TUD-1 catalyst is prepared in the form of 1/16″ extrudates, crushed to 24/36 mesh particles for the lab performance test. The selective hydrogenation is carried out in a tubular reactor of 0.75″ OD. The feed consists of 0.8% methylacetylene, 0.3% propadiene, 22% propylene; and the balance is isobutane. Hydrogen is dissolved in this hydrocarbon stream. The molar ratio of hydrogen/(methylacetylene+propadiene) is about 0.75. This mixture is then sent to the reactor. The LHSV is maintained at approximately 367. At the end of the reaction, conversion and selectivity are measured. Selectivity is defined as the propylene made/[(methylacetylene+propadiene) converted]×100. At 49° C. and 400 psig, (methylacetylene+propadiene) conversion is 29%, and selectivity is 71%.

While the above description contains many specifics, these specifics should not be construed as limitations on the scope of the invention, but merely as exemplifications of preferred embodiments thereof. Those skilled in the art will envision many other possibilities within the scope and spirit of the invention as defined by the claims appended hereto.

What is claimed is:

1. A process for the hydrogenation of a hydrocarbon feed containing unsaturated components, which comprises:
a) providing a catalyst including at least one Group VII metal on a noncrystalline, mesoporous inorganic oxide support having at least 97 volume percent interconnected mesopores based upon mesopores and micropores, having BET surface area of at least 300 m²/g, and a pore volume of at least 0.3 cm³/g; and
b) contacting the hydrocarbon feed with hydrogen in the presence of said catalyst in a hydrogenation reaction zone under hydrogenation reaction conditions to provide a product having a reduced content of unsaturated components.

2. The process of claim 1 wherein the Group VIII metal is a noble metal.

3. The process of claim 2 wherein the noble metal is selected from the group consisting of palladium, platinum, rhodium, ruthenium, and iridium.

4. The process of claim 1 wherein the Group VIII metal is nickel.

5. The process of claim 1 wherein the Group VIII metal has a percentage composition of at least about 0.1 percent by eight based upon the total catalyst weight.

6. The process of claim 1 wherein the mesoporous inorganic oxide support has a BET surface area of from about 400 m²/g to about 1,200 m²/g and a pore volume of from about 0.4 cm³/g to about 2.2 cm³/g.

7. The process of claim 1 wherein the unsaturated components of the hydrocarbon feed comprise aromatics and/or olefins.

8. The process of claim 7 wherein said process is dearomatization.

9. The process of claim 1 wherein the hydrogenation reaction conditions include a temperature of from about 150° C. to about 400° C., a hydrogen partial pressure of from about 200 psi to about 2,000 psi, a LHSV of from about 0.2 hr⁻¹ to about 10.0 hr⁻¹, and a hydrogen circulation rate of from about 500 SCF/Bbl to about 20,000 SCF/Bbl.

10. The process of claim 1 wherein the hydrogenation reaction conditions include a temperature of from about 260° C. to about 650° C., a hydrogen partial pressure of from about 500 psi to about 1,500 psi, a LHSV of from about 0.5 hr⁻¹ to about 3.0 hr⁻¹, and a hydrogen circulation rate of from about 2,000 SCF/Bbl to about 15,000 SCF/Bbl.

11. The process of claim 1 wherein the hydrogenation reaction conditions include a temperature of from about 275° C. to about 330° C., a hydrogen partial pressure of from about 600 psi to about 1,200 psi, a LHSV of from about 1.0 hr⁻¹ to about 2.0 hr⁻¹, and a hydrogen circulation rate of from about 3,000 SCF/Bbl to about 13,000 SCF/Bbl.

12. The process of claim 1 wherein the catalyst further comprises a zeolite.

13. The process of claim 12 wherein the zeolite is selected from the group consisting of FAU, EMT, BEA, VFI, AET, CLO and combinations thereof.

14. The process of claim 12 wherein the amount of zeolite is from about 0.05 wt % to about 50.0 wt % based upon the total catalyst weight.

15. The process of claim 1 wherein the hydrocarbon feed comprises a lubricant base stock.

16. The process of claim 1 wherein the feed contains over about 70% by weight aromatics.

17. The process of claim 1 wherein the feed contains over about 50% by weight aromatics.

18. The process of claim 1 wherein said hydrogenation reaction zone comprises at least one fixed bed of catalyst.

19. The process of claim 18 wherein said hydrogenation reaction zone includes at least first and second spaced apart fixed beds of catalyst, wherein effluent of the first fixed bed is directed into the second fixed bed.

20. The process of claim 19 further including the step of cooling the effluent of the first fixed bed before it enters the second fixed bed.

21. The process of claim 1 further including the step of preheating the feed in a feed/effluent heat exchanger and then heating the feed in a furnace up to a reaction temperature.

22. The process of claim 1 wherein said process comprises dearomatization of an aromatic-containing hydrocarbon feed.

23. The process of claim 1 wherein said process comprises hydrogenation of a hydrocarbon lubricant base stock feed having a bromine number greater than 5 in the presence of superatmospheric hydrogen, to produce a lubricant product having a bromine number less than 3.

24. The process of claim 1, wherein said process comprises selective hydrogenation of acetylenic and/or dienic impurities in a feed that contains at least one monoolefin.

25. The process of claim 1 wherein said process comprises selective hydrogenation of olefinic and/or dienic impurities in a feed that contains at least one aromatic compound.

26. The process of claim 24 wherein said impurity comprises acetylenes and one or more compound containing adjacent double bonds, and said hydrocarbon feed includes a compound containing double bonds separated by at least one single bond.

27. A process for stabilizing a lubricating oil containing unsaturated components which comprises:
(a) hydrocracking in a hydrocracking zone a hydrocarbonaceous feedstock of lubricant viscosity to provide an effluent; and
(b) catalytically hydrogenating in a catalytic hydrogenation zone under superatmospheric hydrogen pressure at least part of the effluent of said hydrocracking zone by contacting at least part of said hydrocracking zone effluent with a catalyst comprising at least one noble metal on a noncrystalline, mesoporous inorganic oxide support having at least 97 volume percent interconnected mesopores based upon mesopores and micropores, having BET surface area of at least 300 m²/g, and a pore volume of at least 0.4 cm³/g to provide a lubricant product having a reduced content of unsaturated components.

28. A process for stabilizing a lubricating oil containing unsaturated components which comprises:
(a) hydrocracking in a hydrocracking zone a hydrocarbonaceous feedstock of lubricant viscosity to provide an effluent; and,
(b) catalytically hydrogenating in a catalytic hydrogenation zone under superatmospheric hydrogen pressure at least part of the effluent of said hydrocracking zone by contacting at least part of said hydrocracking zone effluent with a catalyst comprising nickel on a noncrystalline, mesoporous inorganic oxide support having at least 97 volume percent interconnected mesopores based upon mesopores and micropores, having BET surface area of at least 300 m²/g, and a pore volume of at least 0.4 cm³/g to provide a lubricant product having a reduced content of unsaturated components.
